(19) United States
(12) Patent Application Publication
(10) Pub. No.: US 2014/0142389 A1
(43) Pub. Date: May 22, 2014

(54) SEPARABLE TYPE ENDOSCOPE

(71) Applicant: Samsung Electronics Co., Ltd., Gyeonggi-do (KR)

(72) Inventors: Su-Kwang LIM, Seoul (KR); Mun-Kue PARK, Gyeonggi-do (KR); Jin-Won LEE, Gyeonggi-do (KR)

(73) Assignee: Samsung Electronics Co., Ltd., Gyeonggi-do (KR)

(21) Appl. No.: 14/039,849

(22) Filed: Sep. 27, 2013

(30) Foreign Application Priority Data
Nov. 19, 2012 (KR) 10-2012-0130685

(51) Int. Cl. A61B 1/005; A61B 1/00

(52) U.S. Cl. CPC A61B 1/0052; A61B 1/0057; A61B 1/00119; A61B 1/00114
USPC 600/147

(57) ABSTRACT

An endoscope having a manipulation body and a grip body that are separable from each other is provided. The endoscope includes a pair of rotatable dials provided in the manipulation body, a pair of driving gears that have different gear tooth profiles provided in the manipulation body are coupled with the dials, and a pair of driven gears provided in the grip body which rotate and are engaged with the driving gears, in which the driving gears and the driven gears rotate as the dials are rotated.

### PRIORITY

This application claims priority under 35 U.S.C. §119(a) to Korean Patent Application Serial No. 10-2012-0130685, which was filed in the Korean Intellectual Property Office on Nov. 19, 2012, the entire disclosure of which is hereby incorporated by reference.

### BACKGROUND OF THE INVENTION

1. Field of the Invention

The present invention generally relates to an endoscope, and more particularly, to a separable-type endoscope in which a manipulation body portion of an endoscope and a grip body of an insertion portion inserted into a human body are detachably provided.

2. Description of the Related Art

With the introduction of medical endoscopes, early-detection rates of diseases have improved, and endoscopic surgery has become possible, such that side effects caused by abdominal operations may be prevented. Thus, the employment of endoscopes in the medical field has been increasing.

FIGS. 1 and 2 illustrate a perspective view and a side sectional view of a general endoscope 100, respectively. As illustrated in FIGS. 1 and 2, the endoscope 100 has a joint assembly 113 in an end portion of a flexible insertion tube 111 to allow adjustment of an insertion direction during insertion of the endoscope 100 into a human body. In an end portion of the joint assembly 113, a front end portion body 115 is provided in which an image sensor and a light source (or lens) are mounted. In the other end portion of the insertion tube 111, a grip body 101 is provided. The grip body 101 is connected with an image processing apparatus (not shown) through a separate cable 117 and a connector 119.

In the grip body 101, dials 121a and 121b, buttons 125a, and switches 125b used for direction adjustment of the joint assembly 113 are provided for use during a surgical operation.

The dials 121a and 121b are disposed as a pair to adjust a moving direction of the front end portion body 115 upwardly/downwardly and leftward/rightward along a longitudinal direction of the insertion tube 111. In the grip body 101, a sprocket 131 and a chain 133 are provided to transform rotational movement of the dials 121a and 121b into linear movement. The chain 133 is connected with an end portion of the joint assembly 113 through a separate wire 135. As a result, rotation of the dials 121a and 121b is transformed into linear movement of the wire 135 through the sprocket 131 and the chain 133, and due to the linear movement of the wire 135, the joint assembly 113 bends. Thus, an operator of the endoscope 100 may adjust the moving direction of the distal end portion body 115 during insertion of the insertion tube 111 into the human body.

To fix the moving direction of the joint assembly 113, the dials 121a and 121b are maintained in a still state. Hence, lock levers 123a and 123b corresponding to the dials 121a and 121b are provided.

The buttons 125a for manipulation, such as storing a particular image of video captured with the endoscope in the form of a file, may be provided in the grip body 101. When a surgical operation is performed using the endoscope 100, the valve manipulation switches 125b for inserting and discharging a medicine for cleaning/sterilization may be provided in the grip body 101. Moreover, a treatment hole 127a through which an optical knife necessary for the surgical operation may be provided and a cap 127b for preventing contamination of the treatment hole 127a are provided in the grip body 101.

Meanwhile, since the endoscope is intended to be inserted into a human body, strict health administration is required, and before and after medical treatment or a surgical operation, cleaning/sterilization should be necessarily required. During cleaning/sterilization of the endoscope, it is more cost-efficient to clean only portions which are actually inserted into a human body, that is, the insertion tube and the joint assembly.

Attention needs to be paid to ensure that dials or various switches/buttons provided in the grip body are not contaminated by a cleaning solution.

According to Japanese Patent Publication Gazette No. 2000-014628, an insertion portion and a manipulation portion of an endoscope are configured to be separable to facilitate cleaning/sterilization of the insertion portion.

When the insertion portion and the manipulation portion of the endoscope are separated from each other, a mechanical connector structure capable of delivering manipulation of, for example, a dial to a joint assembly, a channel connector structure for injecting/discharging a medicine for cleaning/sterilization necessary for a surgical operation, and an electric signal connector for control signal transmission to an image sensor and a light source and signal transmission for image information should be disposed in the manipulation portion.

However, disposition of such various connectors in a grip body that is generally handled/manipulated with one hand is accompanied by an increase in the size and weight of the grip body, causing significant fatigue to an operator when the operator performs procedures and surgical operations using the endoscope.

### SUMMARY OF THE INVENTION

The present invention has been made to at least partially solve, alleviate, or remove at least one of problems and/or disadvantages described above, and to provide at least the advantages described below.

Accordingly, aspects of the present invention provide a light-weight endoscope which may be miniaturized and may be configured such that a manipulation body is separable from a grip body.

Aspects of the present invention also provide an endoscope which facilitates separation of a manipulation body from a grip body.

Moreover, aspects of the present invention provide an endoscope which fits a bodily shape of an operator and allows compatibility between a grip body and a manipulation body.

Furthermore, aspects of the present invention provide an endoscope which prevents contamination of a connection portion with a manipulation body in cleaning/sterilization of a separated grip body.

According to an aspect of the present invention, an endoscope having a manipulation body and a grip body that is removable from the manipulation body is provided. The endoscope includes a pair of rotatable dials provided in the manipulation body, a pair of driving gears having different gear tooth profiles provided in the manipulation body and coupled with the dials, a pair of driven gears provided in the grip body configured to rotate and engaged with the driving gears, in which the driving gears and the driven gears rotate in response to rotation of the dials.

### BRIEF DESCRIPTION OF THE DRAWINGS

The above and other aspects, features and advantages of embodiments of the present invention will be more apparent from the following detailed description taken in conjunction with the accompanying drawings, in which:

FIG. 1 is a perspective view showing a general endoscope;

FIG. 2 is a diagram for describing some operations of the endoscope illustrated in FIG. 1;

FIG. 3 is a side view showing a separable-type endoscope according to an embodiment of the present invention;

FIG. 4 illustrates an internal structure of the endoscope illustrated in FIG. 3;

FIG. 5 is a perspective view showing a manipulation body of the endoscope illustrated in FIG. 3;

FIG. 6 is a perspective view showing an enlargement of a coupling portion of a manipulation body illustrated in FIG. 5;

FIG. 7 is a perspective view showing a grip body of the endoscope illustrated in FIG. 3;

FIG. 8 is a perspective view showing an enlargement of a coupling portion of a grip body illustrated in FIG. 7;

FIGS. 9 through 13 are diagrams sequentially showing a process of engagement between gear pairs illustrated in FIG. 4;

FIG. 14 is a perspective view showing a driven gear of the gear pairs illustrated in FIG. 4;

FIG. 15 is a perspective view showing a cap provided in a coupling portion of the grip body illustrated in FIG. 7; and

FIG. 16 is a perspective view showing a state in which the cap illustrated in FIG. 15 is coupled to the coupling portion of the grip body.

Throughout the drawings, like reference numerals will be understood to refer to like parts, components, and structures.

### DETAILED DESCRIPTION OF EMBODIMENTS OF THE PRESENT INVENTION

The following description with reference to the accompanying drawings is provided to assist in a comprehensive understanding of embodiments of the invention as defined by the claims and their equivalents. It includes various specific details to assist in that understanding, but these are to be regarded as mere examples. Accordingly, those of ordinary skill in the art will recognize that various changes and modifications of the embodiments described herein can be made without departing from the scope and spirit of the invention. In addition, descriptions of well-known functions and constructions may be omitted for clarity and conciseness.

The terms and words used in the following description and claims are not limited to their dictionary meanings, but are merely used to enable a clear and consistent understanding of the invention. Accordingly, it should be apparent to those skilled in the art that the following description of embodiments of the present invention is provided for illustration purposes only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

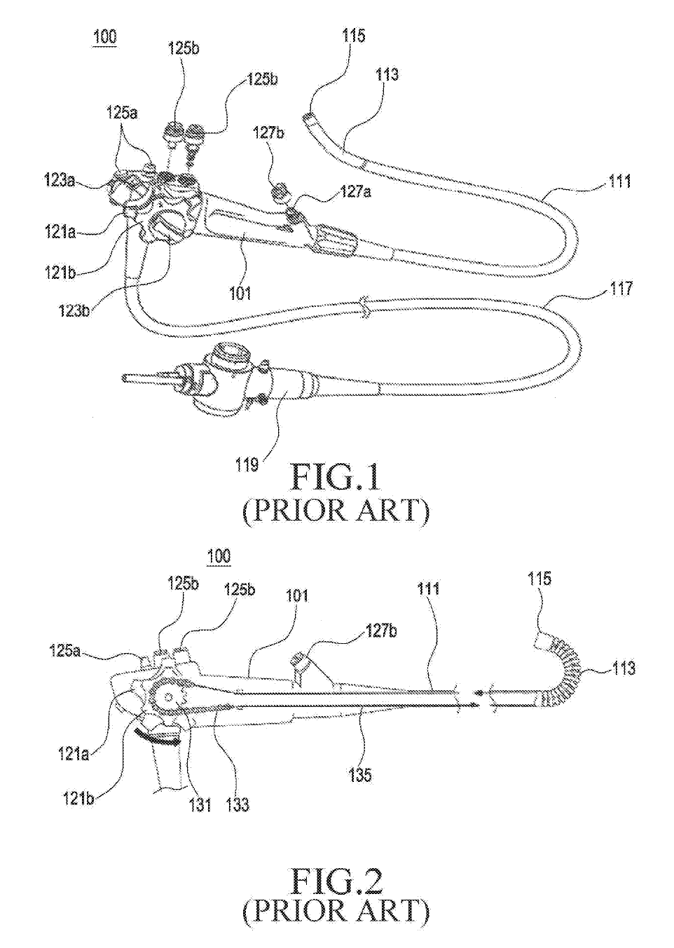

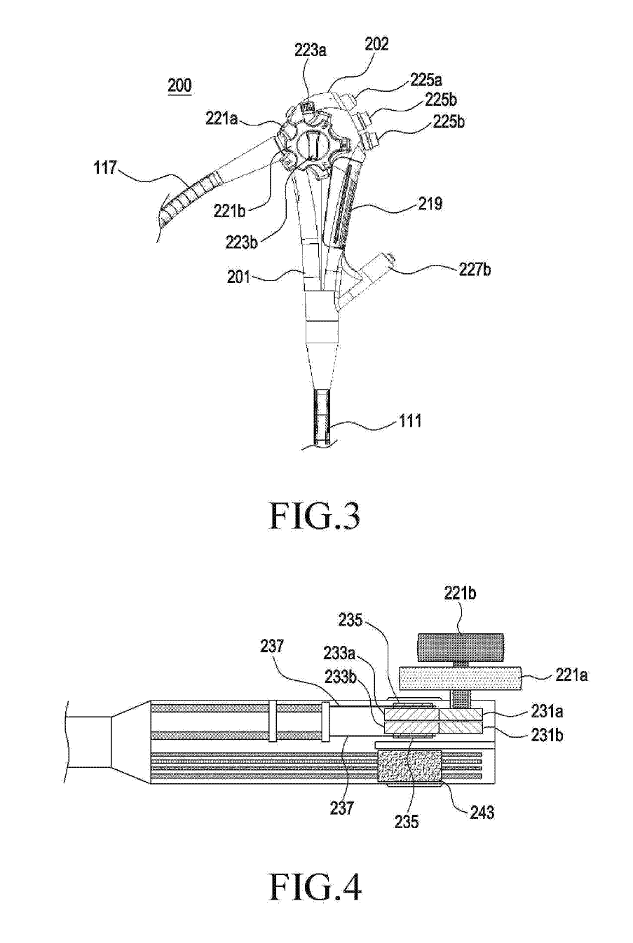

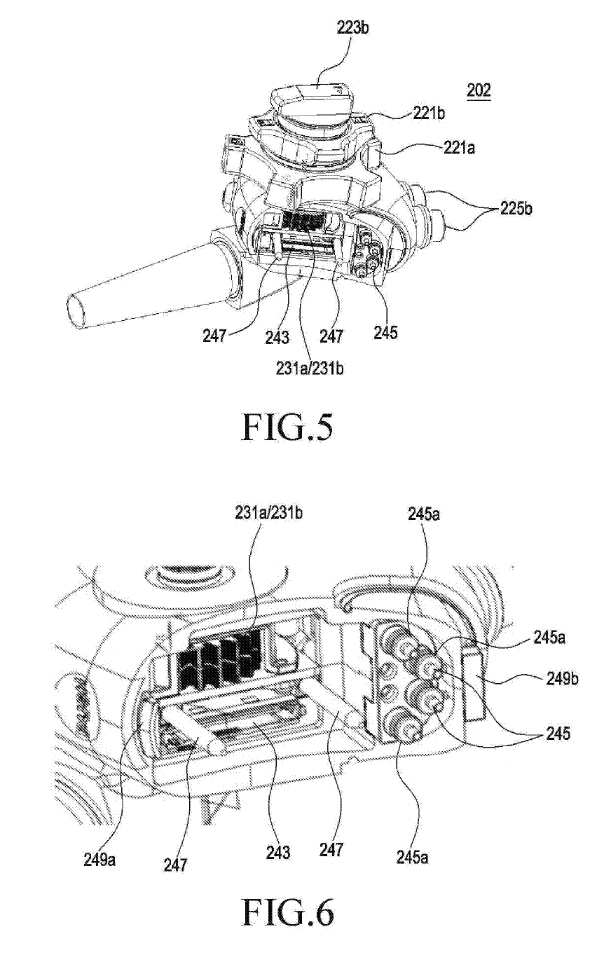

As illustrated in FIG. 3, an endoscope 200 according to an embodiment of the present invention is similar to the general endoscope of FIGS. 1 and 2 in its appearance. However, as illustrated in FIGS. 4 through 8, the endoscope 200 according to the present invention includes a grip body 201 and a manipulation body 202 that are structured to be removably connected to each other, in which mechanical, electrical, and physical connection between the grip body 201 and the manipulation body 202 is implemented through various connectors. In particular, when dials 221a and 221b mounted on the manipulation body 202 are rotated, a mechanical connector using the gear pairs is implemented, thus allowing miniaturization and lightweight construction.

According to an embodiment of the present invention, components such as an insertion tube, a joint assembly, a front end body, a cable, and a connector may be the same as those of the endoscope illustrated in FIG. 1, such that the components will not be described in detail or will be described with reference to FIG. 1.

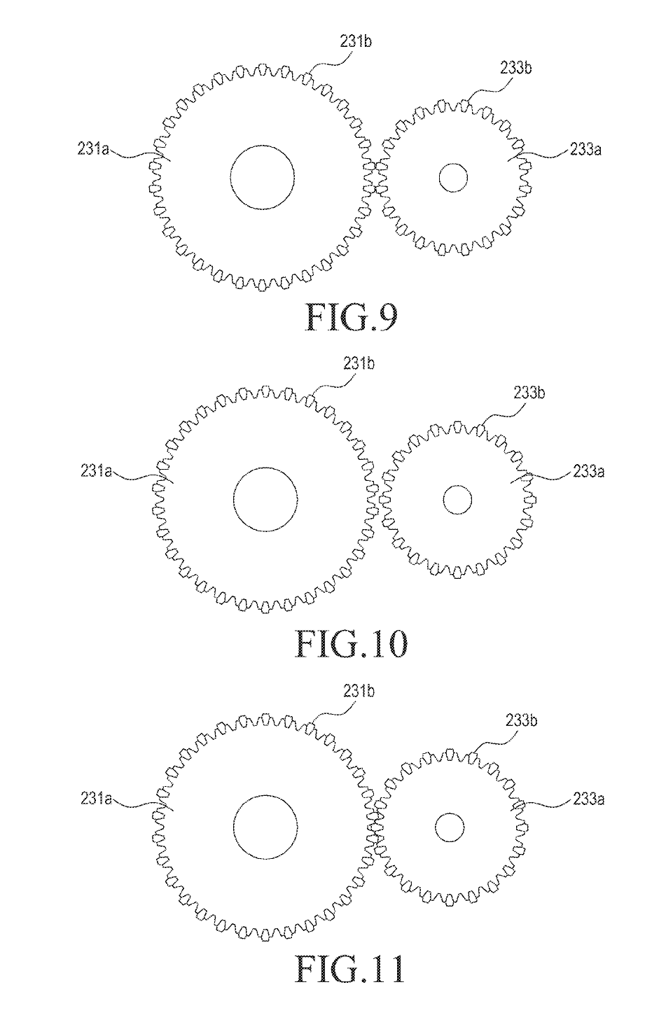

As illustrated in FIGS. 3 through 8, in the endoscope 200, the grip body 201 and the manipulation body 202 are separable and removable from each other, and gear pairs are disposed between the grip body 201 and the manipulation body 202. The gear pairs deliver rotation to the dials 221a and 221b, such that when the endoscope 200 is inserted into the human body, a moving direction of the front end body 115 of the joint assembly 113 is adjusted. In this case, the gear pairs are configured to have different gear tooth profiles. That is, a gear tooth profile of a first gear pair including a first driving gear 231a and a first driven gear 233a, and a gear tooth profile of a second gear pair including a second driving gear 23 lb and a second driven gear 233b, have different sizes and shapes, which easily prevents the top ends of the gear tooth profiles from meeting each other and interfering with each other when the grip body 201 and the manipulation body 202 are coupled with each other. This will be described in more detail with reference to FIGS. 9 through 13.

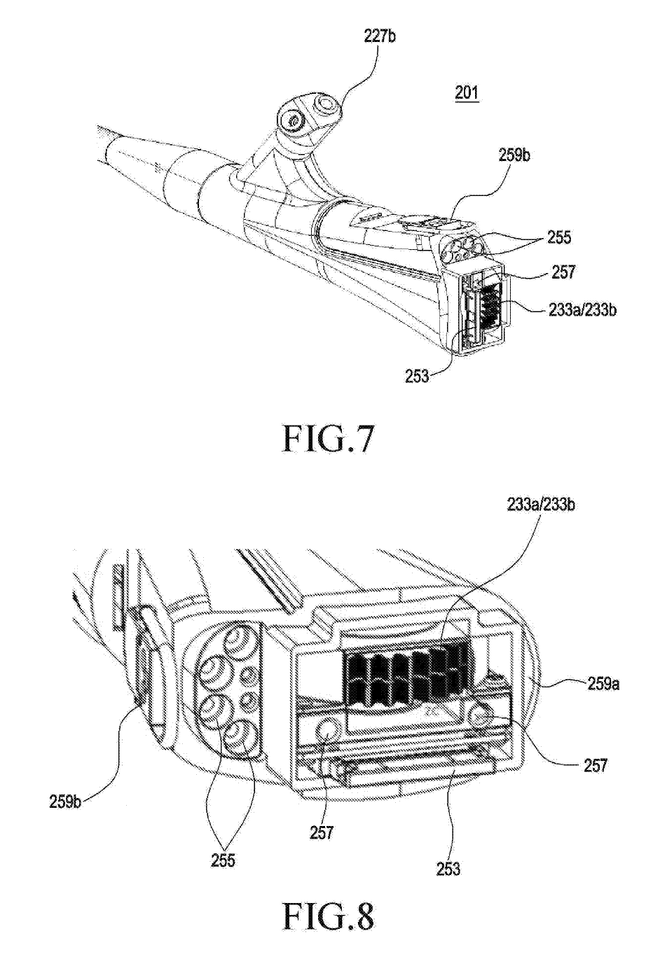

The grip body 201 is provided with a contamination prevention cap 227b for hermetically sealing a treatment aperture, and the manipulation body 202 is coupled to one end of the grip body 201. The insertion tube 111 extends from the other end of the grip body 201, and a wire 237 connected to the joint assembly 113, circuit lines connected to an image sensor or a light source, and various fluid channels for injecting and discharging fluids necessary for cleaning or sterilization are disposed in the insertion tube 111. A cover 219 is coupled to an outer side of the grip body 201. The cover 219 conceals and protects a buckle 259b (FIG. 7) for binding the grip body 201 and the manipulation body 202 together.

The manipulation body 202 is removably provided in one end of the grip body 201 and includes the dials 221a and 221b, valve manipulation switches 225b, and buttons 225a which are installed on an outer side of the manipulation body 202. The dials 221 a and 221b are rotatably installed on the manipulation body 202, and are used to adjust a moving direction of the front end body 115 during an insertion process. The manipulation body 202 includes lock levers 223a and 223b for fixing and holding the dials 221a and 221b in a rotation-stop state. When a surgical operation is performed or a particular region is photographed inside an organ, the front end body 115 needs to be fixed in a particular position. To this end, by fixing the dials 221a and 221b with the lock levers 223a and 223b, movement of the front end body 115 may be suppressed. The valve manipulation switches 225b are intended to manipulate a valve installed in a supply/discharge channel of a fluid when an antiseptic solution/water/air is supplied or the fluid or a foreign substance needs to be discharged around the front end body 115. The buttons 225a are used to capture a still image by the image sensor or control an illumination of the front end body 115.

Since the grip body 201 connected with the insertion tube 111 is separable from the 30 manipulation body 202, an operator may select and use a manipulation body 202 having a size and an arrangement that suitable for her/his body shape or body condition. The operator may also couple a grip body 201 having a different structure for a different diagnosis purpose to the manipulation body 202 while using the manipulation body 202 suitable for his/her bodily shape. That is, an endoscope for the large intestine and an endoscope for the stomach may have different structures, and the operator may select or choose a proper endoscope with the grip body 201 according to the present invention is provided.

Hereinafter, with reference to FIGS. 4 through 8, a coupling portion between the grip body 201 and the manipulation body 202 will be described in more detail. Between the grip body 201 and the manipulation body 202, a mechanical connector including the aforementioned gear pairs, an electric signal connector for transmission of an electric signal, and a fluid channel connector for connecting fluid channels for injecting/discharging a fluid such as medicine are disposed.

The mechanical connector between the grip body 201 and the manipulation body 202 includes first and second driving gears 231a and 231b disposed in the manipulation body 202 and first and second driven gears 233a and 233b disposed in the grip body 201. As mentioned above, the first driving gear 231a and the first driven gear 233a are engaged as a pair, and the second driving gear 23 lb and the second driven gear 233b are engaged as a pair. The first driving gear 231a is coupled to one of the dials 221a and 221b, such as the first dial 221a, and the second driving gear 231b is coupled to the second dial 221b, such that the first driving gear 231a and the second driving gear 231b rotate on the manipulation body 202. In this case, a gear tooth profile of the first driving gear 231 a and a gear tooth profile of the second driving gear 231b are different from each other, and a gear tooth profile of the first driven gear 233a and a gear tooth profile of the second driven gear 233b have shapes corresponding to those of the first driving gear 231a and the second driving gear 231b, respectively.

Cylindrical wire winding portions 235 (FIG. 4) are provided in the first driven gear 233a and the second driven gear 233b, respectively, and wires 237 connected to the joint assembly 113 are partially wound around the wire winding portions 235. Thus, if the first driven gear 233a and the second driven gear 233b rotate, the wires 237 are wound around the wire winding portions 235 along the rotating direction of the first driven gear 233a and the second driven gear 233b. Thus, the joint assembly 113 connected to the wires 237 adjusts a direction of the front end body 115 as the wires 237 are pulled or extended along a direction in which the wires 237 are wound around the wire winding portions 235. Adjustment of the direction of the front end body 115 during insertion of the insertion tube 111 may change the moving direction of the front end body 115. By adjusting the direction of the front end body 115 when the insertion tube 111 is in a stationary state in a particular position, the photographing direction of the image sensor mounted on the front end body 115 may be adjusted.

By including a pair of gear pairs, the user of the endoscope 200 may freely control the direction of the front end body 115. When the pair of the first driving gear 231a and the first driven gear 233a operates, the front end body 115 may be adjusted upwardly or downwardly with respect to the longitudinal direction of the insertion tube 111. When the pair of the second driving gear 231b and the second driven gear 233b operates, the front end body 115 may be adjusted to the left or to the right, perpendicular to the upward or downward direction, with respect to the longitudinal direction of the insertion tube 111. Herein, even when the pair of the first driving gear 231a and the first driven gear 233a operates, the front end body 115 is not necessarily adjusted upwardly or downwardly. Direction adjustment of the front end body 115 is relative, such that the adjustment direction of the front end body 115 that cooperates with the pair of the first driving gear 231a and the first driven gear 233a, and the adjustment direction of the front end body 115 that cooperates with the pair of the second driving gear 23 lb and the second driven gear 233b, are perpendicular to each other. That is, when it is said that the moving direction of the front end body 115 is ‘adjusted upwardly or downwardly/to the left or to the right’, it is merely intended to distinguish relative adjustment directions of the front end body 115 corresponding to operations of the gear pairs.

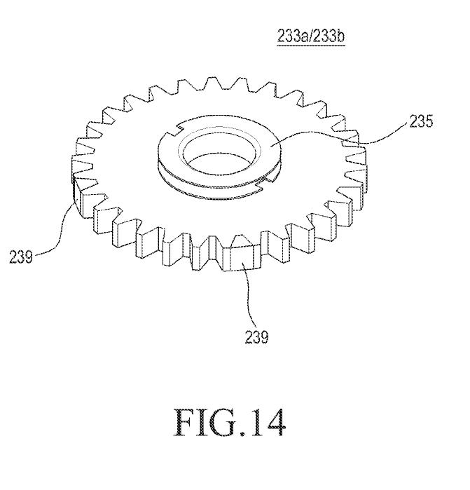

The gears may include a stopper 239 (shown in FIG. 14) for restricting a rotation range of the gears 231a, 231b, 233a, and 233b. If the gears 231a, 231b, 233a, and 233b over-rotate in a particular direction, the joint assembly 113 may be excessively deformed, or an excessive tensile force may be applied to the wires 237 and thus the wires 237 may be cut. Hence, it is desirable to restrict the rotation range of the gears 231a, 231b, 233a, and 233b. Referring to FIG. 14, the first driven gear 233a and the second driven gear 233b include at least one stopper 239 disposed between a pair of gear tooth profiles selected among the gear tooth profiles. As the stopper 239 interferes with a gear tooth profile of a counterpart gear, that is, gear tooth profiles of the first driving gear 231a and the second driving gear 231b, the gears 231a, 231b, 233a, and 233b may not rotate any further. Thus, the user may easily recognize that in manipulation of the dials 221a and 221b, a rotation limit is reached and that the joint assembly 113 is bent to the rotation limit.

Regarding the disposition of the stopper 239, if only one stopper is disposed for one driven gear, the gears 231a, 231b, 233a, and 233b may rotate within an angular range close to 360° . It is apparent that an actual rotation angular range corresponding to disposition of the gears 231a, 231b, 233a, and 233b may vary according to the number of gear tooth profiles and a gear ratio. The rotation range of the gears 231a, 231b, 233a, and 233b may be limited to a smaller range by disposing a pair of stoppers 239 (FIG. 14). If the stoppers 239 are disposed in positions that are spaced apart by about 90°, and are disposed to be engaged with the driving gears 231a and 231b in a circumference having an angular range of 270°, then the driven gears 233a and 233b may rotate about 270°. If the stoppers 239 are disposed in positions spaced apart by about 180°, the driven gears 233a and 233b may rotate in an angular range of about 180°. Disposition of the stoppers 239 and a rotation range of the driven gears 233a and 233b should be set considering an installation structure of the joint assembly 113 and the wire 237.

The electric signal connector includes a plug 253 (FIG. 8) and a socket 243 (FIG. 6) provided between the grip body 201 and the manipulation body 202. The plug 253 and the socket 243 are disposed such that their sides face a side of one of the gears 231a, 231b, 233a, and 233b. The gears 231a, 231b, 233a, and 233b have particular diameters, respectively, and the socket 243 and the plug 253 have specific widths. Thus, when the socket 243 and the plug 253 are disposed adjacent to the gears 231a, 231b, 233a, and 233b, it is preferable that sides of the socket 243 and the plug 253 face the sides of the gears 231a, 231b, 233a, and 233b. To prevent rotation of the gears 231a, 231b, 233a, and 233b from interfering with the socket 243 or the plug 253, a frame forming the grip body 201 or the manipulation body 202 may be interposed between the gears 231a, 231b, 233a, and 233b and the socket 243 or the plug 253. That is, it is desirable to install the gears 231a, 231b, 233a, and 233b and the socket 243 or the plug 253 in independent spaces. The electric signal connector delivers power and control signals to the image sensor or the light source installed in the front end body 115 and an image or an image signal detected from the image sensor, and these signals are generated from or delivered to an image processing apparatus.

The fluid channel connector includes a plurality of female connectors 255 (FIG. 8) and a plurality of male connectors 245 (FIG. 6) which are connected to the fluid channel. The female connectors 255 are disposed in one of the grip body 201 and the manipulation body 202, and the male connectors 245 are disposed in the other of the grip body 201 and the manipulation body 202. In a detailed embodiment of the present invention, the female connectors 255 are disposed in the grip body 201. The fluid channel connector is used to inject or discharge fluids including medicine and discharge foreign substances after a surgical operation. Thus, contamination caused by water leakage and problems with the endoscope equipment may be prevented when the female connector 255 and the male connector 245 are hermetically sealed therebetween. To form a sealing structure between the male connector 245 and the female connector 255, an O-ring 245a may be provided on an inner circumferential surface of the female connector 255 or an outer circumferential surface of the male connector 245. In an embodiment of the present invention, the O-ring 245a is disposed on the outer circumferential surface of the male connector 245.

Meanwhile, when the manipulation body 202 is separated from the grip body 201, the remaining fluid or foreign substances may flow in the female connector 255, the male connector 245, or channels connected to the female connector 255 and the male connector 245, respectively. If the residual substance is a liquid, it may contaminate the periphery of the female connector 255 or the male connector 245. In particular, contamination of the socket 243 and the plug 253 that constitute the electric signal connector may cause serious malfunction or problems with the endoscope equipment, and therefore, it is desirable to prevent contamination of the electric signal connector from occurring due to the residual substance. To this end, as illustrated in FIGS. 6 through 8, the female/male connectors 255 and 245 which constitute the fluid channel connector are disposed stepwise with respect to the socket 243 and the plug 253 which constitute the electric signal connector, thus preventing contamination of the electric signal connector.

For stable connection among the above-mentioned connectors, that is, the mechanical connector, the electric signal connector, and the fluid channel connector, an accurate alignment and coupling path is required during coupling between the grip body 201 and the manipulation body 202. The endoscope 200 according to an embodiment of the present invention includes guide pins 247 and guide holes 257 for guiding the alignment and coupling path of the connectors. The guide pins 247 are disposed in one of the grip body 201 and the manipulation body 202, and extend along a direction of coupling between the grip body 201 and the manipulation body 202. Herein, the phrase ‘the direction of coupling between the grip body 201 and the manipulation body 202’ is substantially the same as a direction of coupling between the socket 243 and the plug 253 which constitute the electric signal connector and a direction of coupling between the female connector 255 and the male connector 245 which constitute the fluid channel connector. The guide holes 257 are formed in the other of the grip body 201 and the manipulation body 202. The guide holes 257 also extend along the direction of coupling between the grip body 201 and the manipulation body 202. In an embodiment of the present invention, the guide pins 247 are formed as a pair in the manipulation body 202, and the guide holes 257 are formed as a pair in the grip body 201, although any number of guide pins 247 will work with a corresponding number of guide holes 257.

When the manipulation body 202 is coupled to the grip body 201, the guide pins 247 are first inserted into the corresponding guide holes 257. As the manipulation body 202 approaches the grip body 201, the guide pins 247 linearly move into the guide holes 257. At this time, the socket 243 and the plug 253 of the electric signal connector and the female connectors 255 and the male connectors 245 of the fluid channel connector have already been aligned for coupling therebetween. Thus, the operator may couple the manipulation body 202 to the grip body 201 without being separately conscious of alignment between the electric signal connector and the fluid channel connector. However, the gear tooth profile of the pair of the driving gears 231a and 231b and the gear tooth profile of the pair of the driven gears 233a and 233b may interfere with each other without being engaged with each other. This may be corrected by manipulating the dials 221 a and 221b during coupling of the manipulation body 202 to the grip body 201, as will be described in detail with reference to FIGS. 9 through 13.

When the manipulation body 202 is coupled to the grip body 201, a means for maintaining the binding state is required. That is, when the endoscope 200 is used, a means for preventing the manipulation body 202 from separating from the grip body 201 and maintaining a stable connection between the connectors is required. The endoscope 200 includes a hook structure and a buckle structure, thus maintaining and fixing coupling of the manipulation body 202 to the grip body 201.

The hook structure includes a hook 249a formed in the manipulation body 202 and a hook ring 259a formed in the grip body 201. The hook 249a is formed in a coupling portion of the manipulation body 202, that is, in a side edge of a surface facing the grip body 201. The hook ring 259a is formed in a position corresponding to the hook 249a in the coupling portion of the grip body 201. When the manipulation body 202 is coupled to the grip body 201, the hook 249a and the hook ring 259a are bound at respective sides of the manipulation body 202 and the grip body 201.

The buckle structure includes a buckle ring 249b formed in the manipulation body 202 and a buckle 259b installed in the grip body 201. The buckle 259b is disposed to linearly move on the grip body 201. When the manipulation body 202 is coupled to the grip body 201, the buckle 259b linearly moves on the grip body 201 to be engaged with the buckle ring 249b. In this case, the buckle 259b is installed in an opposite position to the hook ring 259a, that is, in the other side of the grip body 201. That is, the hook 249a and the buckle 259b are disposed in both-side edges of the coupling portion of the manipulation body 202 or the grip body 201.

If the manipulation body 202 is coupled to the grip body 201, the hook 249a may have already been engaged with the hook ring 259a. On the other hand, the buckle 259b is engaged with the buckle ring 249b only when the operator moves the buckle 259b. That is, only when the buckle 259b is moved to be engaged with the buckle ring 249b, coupling between the manipulation body 202 and the grip body 201 may be completely maintained and fixed. In this state, to prevent erroneous manipulation of the buckle 259b during the use of the endoscope 200, the endoscope 200 includes a separate cover 219, as mentioned with respect to FIG. 3 above. In other words, by concealing and protecting the buckle 259b with the cover 219, unintentional disengagement of the buckle 259b is prevented.

During coupling between the manipulation body 202 and the grip body 201, the gear tooth profile of the first and second driving gears 231a and 231b and the gear tooth profile of the first and second driven gears 233a and 233b may interfere with each other, failing to become engaged with each other. At this time, the endoscope 200 according to the present invention may prevent the gear tooth profiles of the first driving gear 231a and the first driven gear 233a and the gear tooth profiles of the second driving gear 231b and the second driven gear 233b from interfering with each other at the same time by using different gear tooth profiles, in particular, different sizes and shapes, for the first driving gear 231a and the first driven gear 233a and for the second driving gear 231b and the second driven gear 233b.

FIGS. 9 through 13 sequentially illustrate a state in which interference between the gear tooth profiles of the gears 231a, 231b, 233a, and 233b during coupling between the manipulation body 202 and the grip body 201 is addressed, so that the manipulation body 202 and the grip body 201 can be completely coupled. In FIGS. 9 through 13, the gear tooth profiles of the second driving gear 231b and the second driven gear 233b are larger than those of the first driving gear 231a and the first driven gear 233a.

In this case, if the gear tooth profiles interfere with each other during coupling between the manipulation body 202 and the grip body 201, the gear tooth profiles of the second driving gear 231b and the second driven gear 233b may first interfere with each other, as illustrated in FIG. 9. If the operator senses this during coupling between the manipulation body 202 and the grip body 201, the operator rotates the second dial 221b. Thus, as illustrated in FIG. 10, the gear tooth profile of the second driving gear 231b may be adjusted to be engaged with the gear tooth profile of the second driven gear 233b.

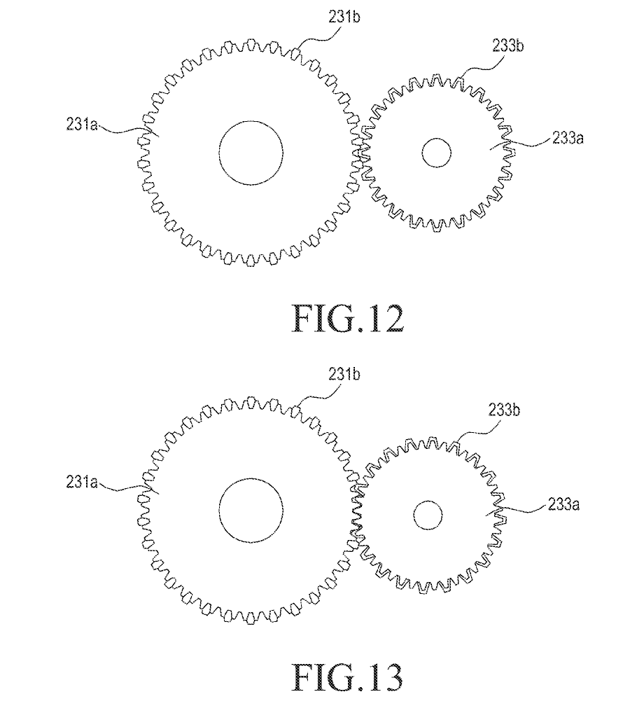

When the gear tooth profile of the second driving gear 231b is adjusted to be engaged with that of the second driven gear 233b, the manipulation body 202 may move closer to the grip body 201. In this process, as illustrated in FIG. 11, the gear tooth profile of the first driving gear 231a may interfere with that of the first driven gear 233a. If the user senses this, the operator rotates the first dial 221a. Hence, as illustrated in FIG. 12, the gear tooth profile of the first driving gear 231a is adjusted to be engaged with that of the first driven gear 233a. Thus, the operator may cause the manipulation body 202 to move directly against the grip body 201, and in this case, the gear tooth profile of the first driving gear 231a and the gear tooth profile of the first driven gear 233a are completely engaged with each other as illustrated in FIG. 13. When the gear tooth profiles of the gears 231a, 231b, 233a, and 233b are completely engaged with each other, the operator engages the hook 249a with the hook ring 259a, and the buckle 259b with the buckle ring 249b and couples the cover 219 to the buckle 259b, such that the manipulation body 202 is completely coupled to the grip body 201.

The separable-type endoscope according to the present invention may stably couple the mechanical connector, the electric signal connector, and the fluid channel connector, and may easily separate them. Thus, various operators may use the grip body and the insertion tube in common, and may select and use a manipulation body according to their particular choice.

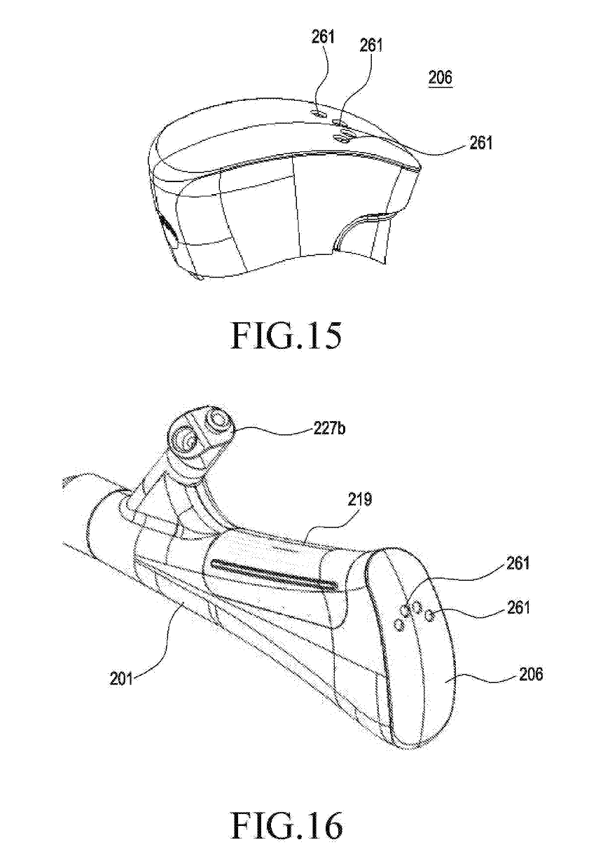

In this case, when the grip body and the insertion tube are used for different patients, it will be obvious that the insertion tube and the joint assembly should be subject to strict health management through thorough cleaning/sterilization. To this end, after the grip body and the manipulation body are separated, the insertion tube and the joint assembly are cleaned and washed. To prevent various connectors of the grip body from being contaminated in cleaning/sterilization, the endoscope 200 may include a separate cap 206, as shown in FIGS. 15 and 16. The cap 206 is coupled to the coupling portion of the grip body 201. In this case, fluids and foreign substances may remain in various channels disposed inside the insertion tube 111 and thus need to be removed. Therefore, the cap 206 includes as many holes 261 as the male connectors 245. When the cap 206 is coupled to the grip body 201, the male connectors 245 are connected to one of the holes 261. Thus, in cleaning/sterilization of the insertion tube 111, the channels in the insertion tube 111 may be cleaned/sterilized through the holes 261.

The endoscope as described above is structured such that the grip body and the manipulation body are separable from each other, and the mechanical connector in the form of a rotatable dial is provided to facilitate miniaturization and light-weight construction. Moreover, by providing the separate cap coupled to the separated grip body, contamination of the grip body and the internal electric connector may be prevented in cleaning/sterilization. Furthermore, during coupling between the mechanical connector, the electric signal connector, and the fluid channel connector, by using the guide pins and the guide holes, stable coupling between the connectors may be induced. In addition, the manipulation body may be replaced with another manipulation body according to the operator's preference, thus alleviating the fatigue of the operator. Since the grip body and the insertion tube connected thereto may be replaced depending on a purpose of use, the cost of the endoscope may be reduced.

While the present invention has been particularly illustrated and described with reference to embodiments thereof, various modifications or changes can be made without departing from the scope of the present invention. Therefore, the scope of the present invention is not limited to the described embodiments, should be defined by the scope of the following claims and any equivalents thereof.

What is claimed is:

1. An endoscope having a manipulation body and a grip body that is removable from the manipulation body, the endoscope comprising:
a pair of rotatable dials provided in the manipulation body;
a pair of driving gears having different gear tooth profiles provided in the manipulation body and coupled with the dials;
a pair of driven gears provided in the grip body configured to rotate and engaged with the driving gears, wherein the driving gears and the driven gears rotate in response to rotation of the dials.

2. The endoscope of claim 1, further comprising:
an insertion tube extending from the grip body;
a joint assembly provided in an end portion of the insertion tube; and
wires connecting the joint assembly with the driven gears,
wherein as the driven gears rotate, the wires move the joint assembly to adjust an insertion direction of the insertion tube.

3. The endoscope of claim 1, further comprising:
at least one guide pin provided in one of the manipulation body and the grip body and extending in a direction of coupling between the manipulation body and the grip body; and
at least one guide hole provided in the other of the manipulation body and the grip body and corresponding to the at least one guide pin,
wherein during coupling of the grip body to the manipulation body, the at least one guide pin is inserted into the at least one guide hole to guide the direction of coupling of the grip body to the manipulation body.

4. The endoscope of claim 1, further comprising an electric signal connector provided between the manipulation body and the grip body.

5. The endoscope of claim 1, further comprising a fluid channel connector provided between the manipulation body and the grip body.

6. The endoscope of claim 5, further comprising an electric signal connector provided between the manipulation body and the grip body,
wherein the fluid channel connector and the electric signal connector are disposed stepwise with respect to each other on one of the manipulation body and the grip body.

7. The endoscope of claim 5, wherein the fluid channel connector comprises:
a male connector provided in one of the manipulation body and the grip body;
a female connector provided in the other of the manipulation body and the grip body; and
an O-ring provided on one of an outer circumferential surface of the male connector and on an inner circumferential surface of the female connector.

8. The endoscope of claim 5, further comprising:
a cap provided on the grip body to be removable from the grip body; and
holes formed in the cap,
wherein the cap is coupled to the grip body to protect the driven gears, and the holes are connected to the respective channels of the fluid channel connector.

9. The endoscope of claim 1, further comprising at least one stopper provided between a pair of gear teeth in a circumference of the driven gear,
wherein the stopper interferes with a gear tooth of the driving gear to limit a rotation range of the driven gear.

10. The endoscope of claim 9, wherein the at least one stopper comprises a pair of stoppers in each driven gear, and the rotation range of the driven gear is limited to 180° or less.

11. The endoscope of claim 1, further comprising:
a hook formed on a side surface of the manipulation body that faces the grip body; and
a hook ring formed on the grip body,
wherein when the manipulation body is coupled to the grip body, the hook is engaged with the hook ring to maintain coupling of the manipulation body to the grip body.

12. The endoscope of claim 11, further comprising:
a buckle ring formed on the other side surface of the manipulation body; and
a buckle installed to linearly move on the grip body,
wherein when the manipulation body is coupled to the grip body, the buckle is engaged with the buckle ring to maintain coupling of the manipulation body to the grip body.
